(19) United States
(12) Patent Application Publication
(10) Pub. No.: US 2007/0093555 A1
(43) Pub. Date: Apr. 26, 2007

(54) Topical pain relief compositions of N,2,3-trimethyl-2-isopropylbutamide and methods for using the same

(76) Inventors: Jutaro Shudo, San Jose, CA (US); Yuji Machida, Kagawa (JP); Seiichiro Kawabata, Kagawa (JP)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/582,130

(22) Filed: Oct. 16, 2006

Related U.S. Application Data
(60) Provisional application No. 60/729,844, filed on Oct. 24, 2005.

(51) Int. Cl. A61K 31/16

(52) U.S. Cl. 514/625

(57) ABSTRACT

Topical pain relief compositions of N,2,3-trimethyl-2-isopropylbutamide and methods for using the same are provided. The subject compositions include a pain relieving effective amount of N,2,3-trimethyl-2-isopropylbutamide in a topical formulation, e.g., a patch, gel, cream or foam. Also provided are methods of using the subject compositions in pain relief applications.

### CROSS-REFERENCE TO RELATED APPLICATIONS

Pursuant to 35 U.S.C. § 119 (e), this application claims priority to the filing date of the U.S. Provisional Patent Application Ser. No. 60/729,844 filed Oct. 24, 2005, the disclosure of which is herein incorporated by reference.

### INTRODUCTION

1. Background of the Invention

An area of on-going research is the development of safer and effective methods for reducing or eliminating pain using transdermal analgesic formulations. Over time, a variety of such analgesic formulations have been developed. These include lotions and ointments containing aspirin or any of a number of non-steroidal anti-inflammatory agents.

However, many current topical pain agents are not entirely satisfactory. For example, opioids can cause strong addiction in patients. NSAIDs can cause various undesirable side effects such as nausea, vomiting, constipation and blood clotting. Local anesthetics can also cause various undesirable side effects, such as skin blistering, slow heart rate and dizziness.

As such, while many of the currently available analgesic formulations reduce pain to some degree, there is, nonetheless, a continued interest in identifying new formulations which provide longer lasting pain relief in a short period of time.

Accordingly, there is continued interest in the development of new topical pain relief agents.

2. Relevant Literature

U.S. Pat. Nos. 4,296,255; 4,296,093; 4,230,688; 4,226,988; 4,193,936; 4,153,679; 4,150,052; 4,070,496; 4,070,449; 4,060,091; 4,059,118; 4,034,109; 4,033,994; 4,032,661; 4,020,153; 5,266,592; 4,459,425; 5,773,410; 6,267,974; 6,592,884; 5,959,161; 6,328,982; 6,359,168; 6,214,788; 5,608,119; 6,769,428; 6,455,080; 6,656,456; 6,821,507; 6,740,311, 6,677,391; 6,497,859; 6,769,428 and 6,719,995; Japanese Patent No. 2004059474; U.S. Patent Application No. 20040067970.

### SUMMARY OF THE INVENTION

Topical pain relief compositions of N,2,3-trimethyl-2-isopropylbutamide and methods for using the same are provided. The subject compositions include a pain relieving effective amount of N,2,3-trimethyl-2-isopropylbutamide in a topical formulation, e.g., a patch, gel, cream, spray or foam. Also provided are methods of using the subject compositions in pain relief applications.

### BRIEF DESCRIPTION OF THE FIGURES

FIG. 1 provides a cross-sectional view of a topical patch preparation according to the invention.

### DESCRIPTION OF THE SPECIFIC EMBODIMENTS

Topical pain relief compositions of N,2,3-trimethyl-2-isopropylbutamide and methods for using the same are provided. The subject compositions include a pain relieving effective amount of N,2,3-trimethyl-2-isopropylbutamide in a topical formulation, e.g., a patch, gel, cream, ointment, spray or foam. Also provided are methods of using the subject compositions in pain relief applications.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,”“only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

In further describing various representative embodiments of the invention, representative topical compositions are reviewed first in greater detail, followed by a discussion of representative methods and applications in which the subject compositions may be used, as well as representative kits of the subject compositions that may be used in the methods of the invention.

Topical Pain Relief Compositions

As summarized above, the subject invention is directed to topical pain relief compositions and methods for their use in treating a subject in need of pain relief, e.g., known to be suffering from pain. A feature of the subject topical pain relief compositions is the presence of a pain relief effective amount of N,2,3-trimethyl-2-isopropylbutamide (also known as WS-23; CAS#51115-67-4). The WS-23 active agent can be produced using any convenient protocol, where representative protocols are described in U.S. Pat. No. 4,296,255. By pain relief effective amount is meant that the amount of the WS-23 active agent present in the composition is sufficient such that, when topically applied to a subject pursuant to the methods of the invention, the subject experiences pain relief, where pain relief is used to refer not only to a complete cessation of pain, but also some measurable decrease in the magnitude of pain, e.g., as measured using the scale reported in the Experimental Section blow. In representative embodiments, the amount of WS-23 active agent in the topical composition ranges from about 0.1 to 30% (w/w); such as from about 2 to 20% (w/w).

The topical composition may be present in a variety of different topical application formats, including, but not limited to: a patch, e.g., including a hydrogel patch; a gel; a cream; a foam; a lotion; a spray; an ointment; a tape; a plaster; etc.

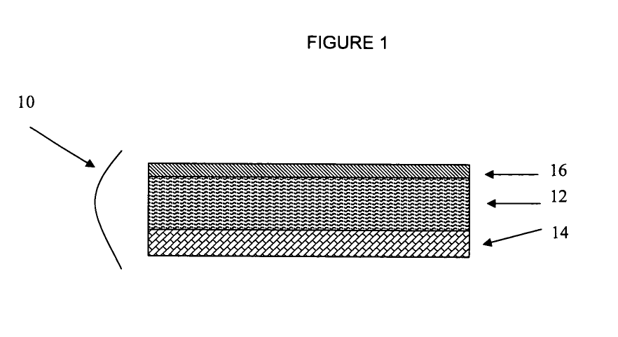

In representative embodiments of the interest, the topical formulation is a topical patch. In certain embodiments, the topical patch preparations of the subject invention are characterized by the presence of a gel adhesive base, and may be viewed as hydrogel patch preparations. As is known in the art, topical patch preparations, such as the embodiment 10 shown in FIG. 1, generally consist of a gel adhesive base 12, a support 13 and a release liner 16. The gel adhesive base is, in representative embodiments, a mixture of (in addition to the active agent) polymers, adhesive resins, solubilizers, thickeners, plasticizers, pH regulators, cross-linking agents, water-retaining agents, preservatives and the like. Furthermore, topical patch preparations may also contain other physiologically acceptable excipients or other minor additives, particularly associated with organoleptic properties, such as fragrances, dyes, emulsifiers, buffers, antibiotics, stabilizers or the like.

The support is generally made of a flexible material which is capable of fitting in the movement of human body and includes, for example, plastic films, various non-woven fabrics, woven fabrics, spandex, and the like.

The release liner is generally made of any convenient material, where representative release films include polyesters, such as PET or PP, and the like.

The topical patch preparation of these representative embodiments may be fabricated using any convenient protocol. One convenient protocol for fabrication of the subject patches includes preparing a gel adhesive base through the uniform mixing of the aforementioned ingredients and then coating the paste onto the support, followed by cutting of the resultant product to the specified size to obtain the desired topical patch preparation. For a more detailed description of the fabrication protocol, see U.S. Pat. No. 5,827,529 and U.S. Application Ser. No. 60/615,320, WO 02/078757 and WO 02/078756 and U.S. Pat. Nos. 5,120,544; 5,160,328; 5,270,358; 5,423,737; 5,476,443; 5,489,262; 5,501,661; 5,827,529; 6,039,940; 6,096,333; 6,214,374; 6,296,869; 6,348,212; 6,455,065; the disclosures of which are herein incorporated by reference.

It should be noted that the above manufacturing protocols are merely representative. Any convenient protocol that is capable of producing the subject topical preparations, as described above, may be employed.

In representative embodiments of the interest, the topical formulation is a gel and cream. As are known in the art. The gel, cream preparations are generally mixture of (in addition to the active agent) water, water soluble polymers, preservatives, alcohols, polyvalent alcohols, emulsifying agents, VASELINE™ petroleum jelly, wax, solvents, thickeners, plasticizers, pH regulators, water-retaining agents and the like. Furthermore, the gel and cream preparations may also contain other physiologically acceptable excipients or other minor additives, particularly associated with organoleptic properties, such as fragrances, dyes, emulsifiers, buffers, antibiotics, stabilizers or the like.

The topical gel and cream preparations of these representative embodiments may be fabricated using any convenient protocol.

In representative embodiments of the interest, the topical formulation is a ointment. As are known in the art. The ointment preparation are generally mixture of in addition to the active agent, wax, VASELINE™ petroleum jelly, preservatives, higher alcohols, polyvalent alcohols, emulsifying agents, solvents, thickeners, plasticizers and the like. Furthermore, the ointment preparation may also contain other physiologically acceptable excipients or other minor additives, particularly associated with organoleptic properties, such as fragrances, dyes, emulsifiers, buffers, antibiotics, stabilizers or the like.

The topical patch preparations of these representative embodiments may be fabricated using any convenient protocol.

Methods of Using WS-23 Topical Compositions

The subject topical compositions find use in applications of treating a subject for pain. As such, the subject compositions find use in methods of treating a subject for pain, where the subject is known to be suffering from pain and the composition is employed to treat the pain.

In practicing the subject methods, the topical composition may be administered to any convenient topical site. Topical sites of interest include, but are not limited to: arms, leg, torso, head, etc. The surface area that is covered by the topical composition following application must be sufficient to provide for the desired amount of agent administration, and in representative embodiments ranges from about 1 to 200 cm², and in many embodiments from about 10 to 180 cm², usually from about 100 to 150 cm², e.g., 140 cm².

In representative embodiments, the period of time that the composition is maintained at the site of application does not exceed about 48 hours, and in representative embodiments does not exceed about 24 hours. However, the period of time during which the preparation is maintained at the application site is, in representative embodiments, at least about 15 to 30 minutes, usually at least about 1 hour.

In practicing the subject methods, a given dosage of the topical composition may be applied a single time or a plurality of times over a given time period, e.g., the course of the pain condition being treated, where the dosing schedule when a plurality of compositions are administered over a given time period may be daily, weekly, biweekly, monthly, etc.

In the subject methods, the topical composition that includes the WS-23 active agent is applied to a keratinized skin site of the subject proximal to site of pain, where the phrase “site of pain” is used to refer to the location of pain as perceived by subject. The site of pain may be present in a variety of body locations. The skin site (i.e., application site) to which the composition is applied will be sufficiently proximal to the site of pain, e.g. the skin site overlies the region of the site of pain, so that upon contact of the composition with the skin surface, the WS-23 active agent can readily reach the site of pain and exert its activity. The particular skin site to which the topical composition is applied will necessarily depend on the location of the site of pain. For example, in treating headache pain, the topical application may be applied to a temple of a subject. Likewise, for treating back pain, the topical composition may be applied to a topical back location of the subject. In representative embodiments, the distance between the site of pain and site of administration does not exceed about 3 cm, and in representative embodiments does not exceed about 1 cm.

The subject compositions are generally applied to the skin site for a period of time sufficient for the desired amount of pain relief to be achieved, where in representative embodiments, the topical composition is applied to the target skin site for a period of time ranging from 0.25 to 24 hours, such as from about 0.5 to 10 hours, including from about 1 to about 8 hours, during which time the subject experiences relief from pain due to the activity of the WS-23 active agent.

If pain recurs following removal of the topical composition, a new topical composition may be applied. The process may be repeated as necessary and desired to achieve pain relief. In representative embodiments, the patient experiences relief from the pain shortly after application. In certain embodiments, the patient will experience at least some relief from the pain about 0.25 to 30 min following application of the topical composition, usually about 5 to 30 min following application of the topical composition.

The amount of composition applied will usually be sufficient to cover a majority of the region of skin overlying the site of pain so that the host experiences pain relief. The exact amount of topical composition that is applied may be determined empirically. For example, the amount of composition applied will be sufficient to cover at least about 50%, more usually at least about 75% of the region. For solutions, dispersions, gels, lotions, creams and the like, the composition may be spread over the region and a covering optionally applied thereto. For patches, an appropriate sized patch may be placed over the region comprising the skin site.

Conveniently, the composition may be provided in a unit dosage format, which formats are known in the art.

Upon application of the topical composition, the WS-23 active agent penetrates the surface of the skin and the subject experiences pain relief. As a result, the patient experiences at least a partial subsidence in the intensity of pain, and in some cases may experience a complete cessation of pain. Thus, application of the topical compositions in accordance with the subject methods results in treatment of the host suffering from pain.

A variety of hosts are treatable according to the subject methods. Generally such hosts are “mammals” or “mammalian,” where these terms are used broadly to describe organisms which are within the class mammalia. Of particular interest is the treatment of primates with the subject methods, (e.g., humans, chimpanzees, and monkeys), where the subject methods are particularly suited for use in the treatment of humans suffering from pain.

In representative embodiments, the subject methods find use in the treatment of a condition characterized by the presence of pain. By treatment is meant at least an amelioration of the pain experience by the subject, where amelioration is used in a broad sense to refer to at least a reduction in the magnitude of a parameter, e.g. pain rating, associated with the pathological condition being treated, side effects associated therewith. As such, treatment also includes situations where the pain is completely inhibited, e.g. prevented from happening, or stopped, e.g. terminated, such that the host no longer suffers from the pain, or at least the symptoms that characterize the pathological condition. In representative embodiment, the subject methods result in a change in magnitude of at least about 1 point as determined on the pain scale reported in the Experimental Section below, such as by at least about 2 points or more, including by at least about 3 points or more, etc. As such, treatment includes both curing and managing a pain condition.

In representative embodiments, the pain condition being treated according to the subject methods is one or more of: back pain, migraine, stiff shoulder, rheumatic arthritis, carpal tunnel syndrome, joint inflammation, bruise, fatigue of a muscle, bone fracture, reflex sympathetic dystrophy, diabetic pain, as reviewed in greater detail below.

Representative Specific Utilities

The present compositions may be used to treat pain associated with many conditions by topically applying the compositions to the area of pain as described above. Specifically, the compositions herein may be used to treat pain, including, but not limited to, arthritis, neck pain, shoulder pain,. back pain, surgical pain, preoperative and postoperative pain, temporal mandibular joint syndrome, carpal tunnel syndrome, and bone injury pain.

The compositions herein may also be used to treat pain associated with osteoarthritis, auto-immune diseases such as rheumatoid arthritis and psoriatic arthritis, gout, psuedo gout, ankylosing spondylitis, juvenile arthritis, systemic lupus erythematosus, arthritis associated with an infection, scleroderma and fibromyalgia.

In addition, the compositions herein may be used to treat muscle pain, pain associated with muscle tension, fatigue, curvature of the spine, minor and major spinal disc compression, pinched nerves, strained or sprained muscles, and nervous tension.

Moreover, the present compositions may be used to treat pain associated with traumatic injuries, hematomas, myositis, lower back syndromes, spinal stenosis, joint pain, bone pain and bone fractures caused by metastic cancer, such as breast, lung. The present composition may also be used to treat muscle, bone and joint pain generally associated with cancer.

The present compositions may be used to treat pain associated with osteoprotic fractures of the lumbar spine and other sites, and traumatic bone fractures, including pelvic fractures. With respect to joint pain, the compositions herein may be used to decrease overall joint stiffness and increase joint mobility.

The present compositions may also be used to treat pain associated with pre-surgical and post-surgical orthopedic procedures. For example, the present compositions may be applied to treat such pain before or after arthroscopy, especially in the shoulders or knees.

In addition, the present compositions may be used for treating pain associated with post-surgical orthopedic recovery, such as tendon, muscle and bone repair, as well as joint replacement, including hip or knee replacement. For example, bone fractures require the use of plates, screws or other attachment means to hold the bones together. Placement of these devices requires surgery, and the post-surgical pain resulting therefrom can be treated with the present compositions.

Further, the compositions herein may be used to treat pain caused by herniated nucleus pulposus (slipped disc), musculo-skeletal pain, joint dislocations, herniated intervetebral disc, prolapsed intervetebral disc (including lumbar and cervical), ruptured disc, whiplash injuries, fibromyositis, intercostal rib pain, muscle tear, tendonitis, bursitis, meniscal tears, tendon tears, and bone spurs. The compositions herein may also be used to treat pain such as cervical muscle hyperactivity (spasm), an extremely common condition with many causes, including tension, response to an inflamed or subluxed joint, arthritic changes, poor posture or work habits, trauma, systemic disease and adjacent pathology.

The compositions of the present invention may be used to treat pain caused by sports related injuries. Such sports-related injuries include, but are not limited to, hematomas, bruises, sprains (e.g., ankle sprain), muscle spasms (e.g., pulled muscles), partial tendon tears, tendonitis, bursitis, myositis, traumatic arthritis and post-insertion of joint dislocation. In treating pain associated with sports related injuries, the present compositions would be applied to the area of pain as described herein. The present compositions may be used in combination with sports-injury therapy techniques such as physical therapy, acupuncture, weight-training, biofeedback techniques, among others.

The present compositions may also be used in treating pain unique to senior citizens. Much of the bone, joint or muscle pain experienced by seniors results from a combination of sources. Some of these sources are known, others are not. In certain cases, such pain is a natural consequence of the diseases resulting from the aging process, which includes pain accompanied with diminished motor function, atrophy, dietary changes, among others. Consequently, pain management in seniors is difficult. Often times, seniors are required to take multiple medications daily in order to effectively manage their pain. This poses significant drawbacks to seniors, such as side effects from the medications, adverse reactions in mixing the medications, as well as excessive costs and effort to maintain the required medication regimen on a daily basis.

Thus, using the present compositions to treat bone, joint or muscle pain in seniors can be effective in minimizing the amount of pain relief medication they already take, or would be required to take in the future. Also, pain in seniors contributes to depression, inactivity and immobility in this age group. Diminution in pain resulting from use of the present compositions would result in greater independence, increased activity, socialization, appetite and overall sense of well-being in an elderly patient.

In addition, the compositions of the present invention can be utilized as an adjunct to physical therapy. Generally, physical therapy involves passive and active treatments or methodologies to strengthen and/or heal muscles, tendons, bones, and joints. The draw backs of physical therapy include pain and discomfort to the patient. The formulations of the present invention can be used to treat such pain. For example, the present formulation may be applied to the area of pain (as described herein) before, during, and/or after each physical therapy treatment.

The present compositions can also be used to treat pain associated with immobilized tissue. Treatment of damaged muscles, bones, tendons, and joints often requires that tissues be immobilized for an extended period of time. In these circumstances, the tissue is kept immobilized by a variety of devices including, but not limited to, braces, slings, casts, bandages and splints. Oftentimes, when the device is removed and continuing thereafter, the patient experiences muscle, bone, tendon and/or joint pain in or about the immobilized area. The present formulation can be used to treat such pain by applying the formulation to the area of pain in the manner described herein.

TENS or transcutaneous electro-nerve stimulation is characterized by high voltage, sensory current and is used to block pain. The present compositions can be used in conjunction with electrical neuromuscular stimulation to increase the effectiveness of the pain treatment. For example, before or after treatment with electrical neuromuscular stimulation, the present composition can be applied to the affected area in the manner described herein.

The present composition can also be used in combination with local or other injections of an anesthetic, such as lidocane (with and without steroids). For example, a needle containing lidocane (with or without a steroids) can be injected into the skin overlying the area of pain. This area of the skin can be further anesthetized by applying the present composition at or around the injection site before or after the injection.

In addition, the present composition may be used in combination with oral analgesics or anti-inflammatories (e.g., NSAIDS and Cox-2-inhibitors) to alleviate pain. When used in such manner, for example, the composition herein can provide an enhanced and/or additive pain relief effect.

The present composition may also be used in combination with heat treatment devices including, but not limited to, hot packs such as heating pads or hot towels. Such devices may also include Diathermy which is a deep tissue heat treatment, wherein the temperature of the injured tissues is raised by high frequency current, ultrasonic waves, or microwaves radiation. Diathermy is used to reduce pain, relieve muscle spasm, decrease soft-tissue contractures, resolve inflammation, and promote healing. The present compositions can be used in combination with hot packs or Diathermy to provide an enhanced and/or additive relief effect.

Further, the present composition may be used in combination with morphine-like agents, such as codeine, opiates, oxy-cotcontin, Percocet, Demorol, and Vicadin. When used in such manner, for example, the morphine-like agents, together with any of the formulations of the present invention, can achieve an analgesic effect that would otherwise require a higher dosage of opioids but with fewer side effects.

In addition, the present composition may be used in combination with biofeedback techniques. Biofeedback is a useful technique for achieving stress reduction, reducing anxiety and alleviating psychosomatic symptoms by monitoring and controlling certain physiological processes. The use of biofeedback techniques in combination with the compositions herein may allow the patient to achieve greater control over his or her physiological processes and to achieve greater reduction in pain than through the use of such techniques.

The present compositions can also be used in combination with acupuncture therapy. Acupuncture therapy generally involves inserting tiny needles at certain specific points on the surface of the body. Acupuncture has proven efficacy in relieving pain. Acupuncture may also be useful for the treatment of osteoarthritis, low back pain, carpal tunnel syndrome, fibromyalgia, and other conditions that cause chronic pain. The compositions herein may provide an enhanced and/or additive relief effect when used in combination with acupuncture.

Kits

Also provided are kits, where the subject kits at least include one or more topical compositions or preparations, as described above. The subject topical preparations in the kits may be present in a package, as described below. Where desired, the topical composition of the kits may be present in individual pouches or analogous containers, to preserve the compositions until use.

The subject kits may also include instructions for how to use the patches, where the instructions typically include information about where to apply the patch, dosing schedules etc. The instructions are generally recorded on a suitable recording medium. For example, the instructions may be printed on a substrate, such as paper or plastic, etc. As such, the instructions may be present in the kits as a package insert, in the labeling of the container of the kit or components thereof (i.e. associated with the packaging or subpackaging) etc. In other embodiments, the instructions are present as an electronic storage data file present on a suitable computer readable storage medium, e.g. CD-ROM, diskette, etc.

The following practical and comparative examples are offered by way of illustration and not by way of limitation.

### EXAMPLES

Practical and comparative examples are given below, but the manufacturing method is not limited thereby.

### Example 1

### Treatment of Back pain with WS-23 Topical Patch Preparation

A. Patch Preparation

A topical 5% WS-23 patch preparation was made as follows: For preparation of the gel adhesive base, 5% WS-23 is mixed well with 12% Castor oil, 0.15% Methyl paraben, 8% Sodium polyacrylate, 0.4% Tartaric acid, 3% Polyvinyl alcohol, 0.04% Aluminum cross-linking agent, 4% Cellulose gum, 20% Glycerin and 47.41% water.

The resultant gel adhesive base is then spread onto a PET nonwoven fabric to a weight of 1000 g/m². The resulting product is then laminated with a PP film and then cut into 10 cm×14 cm.

B. Protocol

The topical patch preparation was applied to lower back of patients for 8 hours/a day for 4 weeks.

C. Results

| | Pain Level | | | | |
|---|---|---|---|---|---|
| Patient Initial | Day 0 | Day 7 | Day 14 | Day 21 | Day 28 |
| Y. M. | 06 | 05 | 05 | 04 | 04 |
| M. S. | 04 | 03 | 04 | 02 | 02 |
| A. N. | 04 | 03 | 02 | 02 | 01 |

*Pain Level:
10: Disabling, Must take care of pain.
08: Severe, Can't concentrate and can't do all but simple things.
06: Moderate, But able to continue some physical activity.
04: Tolerate, Can be ignored somewhat.
02: Mild, Aware of undercurrent of mild pain.
00: Pain Free

### Example 2

### Treatment of Stiff Shoulder

A. Topical Gel Preparation

A topical 20% WS-23 transdermal gel preparation was made as follows: 20% WS-23 was mixed well with 20% Deet, 0.15% Methyl paraben, 20% Water, 5% Carbomer and 34.85% Ethanol, The resultant mixture was then put into a tube and sealed.

B. Protocol

The topical gel preparation was applied to right or left shoulder of patients for two times a day for 8 hours at a time for a week.

C. Results:

Pain Level

| Patient Initial | Day 0 | Day 7 |
|---|---|---|
| H. T. | 08 | 05 |
| T. S. | 06 | 05 |
| M. H. | 06 | 04 |

The scale employed above is the same scale that is employed in 1.C. (above)

### Example 3

### Treatment of Carpal Tunnel Syndrome

A. Topical Cream Preparation

A topical 20% WS-23 transdermal cream preparation was made as follows: 20%WS-23 was mixed well with 40% Propylene Glycol, 7% Water, 0.2% Methyl paraben, 0.1% Propyl paraben, 3% Wax, 1.7% Glyceryl monostearate, 1.3% Hydrogenated castor oil, 1% Polysorbate, 2% Isopropyl myristate, 7% Stearyl alcohol and 16.7% VASELINE™ petroleum jelly. The resultant mixture was then put into a tube and sealed.

B. Protocol

The topical cream preparation was applied to the back of patient's hand for two times a day for 8 hours at a time for 2 weeks.

C. Results

Pain Level

| Patient Initial | Day 0 | Day 7 | Day 14 | Day 21 |
|---|---|---|---|---|
| M. I | 06 | 05 | 05 | 05 |
| S. T. | 05 | 04 | 04 | 03 |
| H. M. | 07 | 04 | 04 | 04 |

The scale employed above is the same scale that is employed in 1.C. (above)

### Example 4

### Treatment of Migraine

A. Topical Ointment Preparation

A topical 20% WS-23 topical ointment preparation was made as follows: 20% WS-23 was mixed well with 25% Deet, 10% Stearyl alcohol, 10% Bees wax and 60% VASELINE™ petroleum jelly. The resultant mixture was then put into a tube and sealed.

B. Protocol

The topical ointment preparation was applied to each temple of the subject for 8 hours/day for 2 days.

C. Results

| | Pain Level | | |
|---|---|---|---|
| Patient Initial | Day 0 | Day 1 | Day 2 |
| A. O. | 04 | 01 | 00 |
| Y. K | 05 | 03 | 01 |
| M. O. | 05 | 02 | 00 |

The scale employed above is the same scale that is employed in 1.C. (above)

It is evident from the above results and discussion that the subject invention provides an important new topical pain relief active agent, which composition offers benefits over currently employed topical active agents, including lack of side effects. As such, the subject invention represents a significant contribution to the art.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A topical pain relief composition comprising a pain relieving effective amount of N,2,3-Trimethyl-2-Isopropylbutanamide.

2. The composition according to claim 1, wherein said topical composition comprises from about 0.1 to 30% N,2,3-Trimethyl-2-Isopropylbutamide.

3. The composition according to claim 2, wherein said topical composition comprises from about 2 to 20% N,2,3-Trimethyl-2-Isopropylbutamide.

4. The composition according to claim 1, wherein said topical composition is a topical patch.

5. The composition according to claim 4, wherein said topical patch is a hydrogel patch.

6. The composition according to claim 1, wherein said topical composition is a gel.

7. The composition according to claim 1, wherein said topical composition is a cream.

8. The composition according to claim 1, wherein said topical composition is a foam.

9. A method treating pain in a subject experiencing pain, said method comprising topically administering to said subject a topical pain relief composition comprising an amount of N,2,3-Trimethyl-2-Isopropylbutanamide effect to treat said subject for said pain.

10. The method according to claim 9, wherein said topical composition is topically administered to said subject at a location proximal to said pain.

11. The method according to claim 9, wherein said topical composition comprises from about 0.1 to 30% N,2,3-Trimethyl-2-Isopropylbutamide.

12. The method according to claim 11, wherein said topical composition comprises from about 2 to 20% N,2,3-Trimethyl-2-Isopropylbutamide.

13. The method according to claim 9, wherein said topical composition is a topical patch.

14. The method according to claim 13 wherein said topical patch is a hydrogel patch.

15. The method according to claim 9, wherein said topical composition is a gel.

16. The method according to claim 9, wherein said topical composition is a cream.

17. The method according to claim 9, wherein said topical composition is a foam.

18. The method according to claim 9, wherein said pain is chosen from the group consisting of back pain, minor sport injury, migraine, stiff shoulder, rheumatic arthritis, carpal tunnel syndrome, joint inflammation, bruise, fatigue of a muscle, bone fracture, reflex sympathetic dystrophy and diabetic pain.

19. The method according to claim 9, wherein said subject is a mammal.

20. The method according to claim 19, wherein said mammal is a human.

21. A kit comprising:
(a) a topical pain relief composition comprising an pain relieving effective amount of N,2,3-Trimethyl-2-Isopropylbutanamide; and
(b) instructions for using said composition to provide pain relief to a subject in need thereof.

22. The kit according to claim 21, wherein said kit comprises a plurality of said compositions.

23. The kit according to claim 22, wherein said plurality of compositions are present in separate containers.

24. The kit according to claim 23, wherein said separate containers are sealed pouches.
